(19) United States
(12) Patent Application Publication
(10) Pub. No.: US 2011/0177493 A1
(43) Pub. Date: Jul. 21, 2011

(54) USING HIGHLY SENSITIVE SUSPENDED CARBON NANOTUBES FOR MOLECULAR-LEVEL SENSING BASED ON OPTICAL DETECTION

(75) Inventor: Jennifer Lu, Merced, CA (US)

(73) Assignee: The Regents of the University of California

(21) Appl. No.: 12/371,163

(22) Filed: Feb. 13, 2009

Related U.S. Application Data
(60) Provisional application No. 61/029,254, filed on Feb. 15, 2008.

(51) Int. Cl. C12Q 1/70; B01J 31/12; C01B 31/00; C07D 495/04; C07K 17/14; C07H 21/04; G01N 33/50; G01N 33/53; H01L 21/02; B82Y 15/00; B82Y 30/00

(52) U.S. Cl. 435/5; 502/155; 423/448; 548/303.7; 530/409; 536/23.1; 436/94; 436/86; 435/7.1; 428/141; 422/68.1; 438/1; 977/745; 977/742; 977/752; 977/751; 257/E21.002

(57) ABSTRACT

A molecular sensor is provided that contains at least one carbon nanotube suspended on a suitable support structure. In one aspect, at least one receptor is attached to a surface of the suspended carbon nanotube. Also provided are methods of detecting an analyte in a sample by contacting a sample suspected of containing the analyte with the molecular sensor of this invention under suitable conditions that favor binding of the analyte to the receptor and detecting any analyte bound to the receptor, if present.

### CROSS-REFERENCE TO RELATED APPLICATIONS

This invention claims priority under 35 U.S.C. §119(e) to U.S. Provisional Application No. 61/029,254, filed Feb. 15, 2008, the contents of which is hereby incorporated by reference into the present disclosure.

### FIELD OF THE INVENTION

This invention relates to the field of molecular sensors, and more particularly, to the use of modified suspended carbon nanotubes to detect analytes.

### BACKGROUND OF THE INVENTION

Throughout this disclosure, various technical and patent publications are referenced to more fully describe the state of the art to which this invention pertains. These publications are incorporated by reference, in their entirety, into this application.

Nanotubes are typically, but not exclusively, carbon molecules and have novel properties that make them potentially useful in a wide variety of applications in electronics, optics, optoelectronics, biological sensing and drug delivery. They exhibit extraordinary strength and unique optical and electrical properties, and are efficient conductors of heat. The name is derived from their size, since the diameter of a nanotube can be on the order of a few nanometers (approximately 50,000 times smaller than the width of a human hair), while they can be up to several centimeters in length. There are two main types of nanotubes: single-walled nanotubes (SWNTs) and multi-walled nanotubes (MWNTs). Bulk synthesized nanotubes naturally group into “ropes” due to strong Van der Waals forces.

Carbon nanotubes (CNTs) are generally produced by three main techniques, arc discharge, laser ablation and chemical vapor deposition. The arc discharge method involves a carbon vapor as a precursor to CNTs that is created by an arc discharge between two carbon electrodes. The carbon nanotubes can be synthesized from the resulting carbon vapor. The laser ablation technique involves a high-power laser beam coming in contact with a volume of carbon-rich gas (methane or carbon monoxide, for example). Laser ablation produces a small amount of clean nanotubes, whereas arc discharge generally produces large quantities of impure CNTs. Chemical vapor deposition (CVD) often utilizes a catalyst nanoparticle and two gases which are bled into the reactor: a process gas (such as ammonia, nitrogen, hydrogen, etc.) and a carbon-containing gas (such as acetylene, ethylene, ethanol, methane, etc.). Nanotubes grow at the sites of catalyst nanoparticle as the carbon-containing gas is broken apart. It is generally believed that oversaturated carbon is diffused to the edges of the catalyst particle wherein this leads to the growth of CNTs. The catalyst particles either stay at the tips of the growing nanotube during the growth process, or remain at the nanotube base, depending on the adhesion between the catalyst particle and the substrate on which the catalyst particle is disposed. Generally, MWNTs and SWNTs that are produced by chemical vapor deposition have a large, poorly controlled diameter range. In order to control the diameter of the CNTs in general, one must use uniformly sized catalyst particles. In addition, the nanotube growth sites can be controlled by careful deposition of the catalyst particles at predefined locations.

Molecular sensors are transducers (sensing elements) which can detect the presence of and change in the concentration of an analyte molecule. Although many types of molecular sensors are known, better sensors would be valuable in many situations. Although molecular sensors have been proposed, none are commercially available. A robust, reproducible and simple detection method is desirable. In environmental protection, various pollutants and effluents from factories and human waste must be monitored more thoroughly in all states of matter, such as the atmosphere, ground and ocean water and soil. For defense, new ways to detect explosives and chemical and biological agents are immediately required in areas such as airports, train stations and other public arena in order to ensure public safety. There is therefore a need for analyte sensors that can detect the presence and concentration of a wide variety of analytes under a variety of environmental conditions, such as atmospheric gas and liquid samples.

The size of carbon nanotubes is comparable to that of biological molecules such as DNA (2-3 μm in diameter). Nanotubes have been utilized as molecular sensors to a limited extent in both fluorescence and conductance-based sensing systems. However, currently designed sensing schemes using carbon nanotubes (CNTs) are less than ideal. Known fluorescence-based CNTs molecular sensors utilize CNTs which are suspended in solution. Often, a surfactant is used to prevent the formation of bundles in solution, but this may quench the fluorescent signal and limit sites on CNTs available for the incorporation of a surface-bound receptor. Functionalization of the CNTs can also be used as a method to prevent bundle formation. However, this also may limit the number of sites available for the incorporation of a surface-bound receptor and, in addition, can alter the fluorescence inherent to the nanotubes.

Known conductance-based CNTs molecular sensors require high purity, defect-free pure semi-conducting CNTs. The current CNT synthesis methods cannot yield all semi-conducting CNTs. In addition, all published sensing methods based on either electronic or acoustic sensing use more complicated architectures involving multiple fabrication steps that can add additional defects to CNTs. The methods used to isolate semi-conducting tubes, such as chemical reagents and radiation, may damage the semi-conducting tubes causing measurement inaccuracy and limited sensitivity. Thus, a need exists for sensitive, reproducible and accurate CNT detection systems. This invention satisfies this need and provides related advantages as well.

### SUMMARY OF THE INVENTION

A molecular sensor is provided, comprising at least one carbon nanotube suspended on a suitable support structure, the suspended carbon nanotube having at least one receptor attached to a surface of the suspended carbon nanotube. The diameter of the suspended nanotube is less than about 2 nm. The CNT is, in one aspect, grown in situ on the suitable support.

Thus, in one aspect this invention provides a molecular sensor comprising, or alternatively consisting essentially of, or yet further consisting of, at least one carbon nanotube as a transducer, based on measuring delta of atomic vibration of Raman sensitive modes upon environmental perturbation, wherein a receptor is first attached to the surface of said carbon nanotube.

In another aspect, the molecular sensor comprises, or alternatively consists essentially of, or yet further consists of, at least one surface-modified suspended carbon nanotube, wherein contact of an analyte to the surface bound receptor changes the carbon nanotube vibrational properties, and the shift of vibration modes can be detected by Raman spectroscopy. For the purpose of illustration only, the vibrational modes are radial breathing mode and/or in-plane graphene vibration.

In a further aspect, the molecular sensor of this invention comprises, or alternatively consists essentially of, or yet further consists of, a 3-dimensional suspended carbon nanotube network which serves to improve the detection capability of said sensor.

Various receptors can be attached to the nanotubes. For the purpose of illustration only, the receptor can comprise, or alternatively consist essentially of, or yet further consist of, an antibody or a ligand. The receptors can further contain labels such as fluorescent molecules that assist in detecting binding of ligand to receptor. The receptor/nanotube structure is useful as a molecular sensor. Thus, in a further aspect, the invention provides a molecular sensor comprising at least one carbon nanotube suspended on a suitable support structure and having at least one receptor attached to a surface of the suspended carbon nanotube. One or more receptors can be attached to the nanotube by any suitable method known in the art such as by Van der Waals forces, ionic bonds, covalent bonds, hydrogen bonds or any other chemical bonds or methods.

More than one of the above described nanotube can be suspended on the support. As such, the invention provides a plurality of molecular sensors positioned on the support. The nanotubes can have an average diameter of less than about 2 nm.

Various receptors can be independently attached to the plurality nanotubes and the receptors can be the same (identical) or different from each other. The receptors can further contain labels such as fluorescent molecules that assist in detecting binding of ligand to receptor. When receptors are attached to the plurality of nanotubes, the nanotubes are positioned proximate to each other to allow binding of at least one receptor to its binding partner. For the purpose of illustration only, the receptor can comprise, or alternatively consist essentially of, or yet further consist of, an antibody or a ligand. The receptor/nanotube structure is useful as a molecular sensor. Thus, in a further aspect, the invention provides a molecular sensor comprising at least one carbon nanotube suspended on a suitable support structure and having at least one receptor attached to a surface of the suspended carbon nanotube. One or more receptors can be attached to the nanotube by any suitable method known in the art such as by Van der Waals forces, ionic bonds, covalent bonds, hydrogen bonds or any other chemical bonds or methods.

Compositions and methods for making the above-noted molecular sensors are also provided. To that end, included in the present invention is a solid support having an indentation with walls substantially perpendicular to the surface of the solid support, the substantially perpendicular walls being coated with a solid oxidic layer and over-coated with a layer of a catalyst-particle-containing polymer. A molecular sensor disclosed herein comprises, or alternatively consists essentially of, or yet further consists of, an oriented carbon-nanotube-containing-solid, wherein a plurality of carbon nanotubes are grown in situ on the particles of the carbon-nanotube-promoting catalyst present in the layer of catalyst on the substantially perpendicular wall of the indentations. Further, at least a portion of the carbon nanotubes bridge the substantially perpendicular walls of an indentation, and still further, at least one receptor is attached to a surface of the carbon nanotubes. More than one receptor can be bound to the singular or the plurality of nanotubes. The receptors can be the same (identical) or different from each other. The indentations can be of any suitable shape or size. For the purpose of illustration, the indentation is a trench or a well. In one aspect, the indentation has a bottom surface substantially parallel to the surface of the solid support and wherein the bottom surface is not coated with the solid oxidic layer. In another aspect, the indentation has a bottom surface substantially parallel to the surface of the solid support and comprises, or alternatively consists essentially of, or yet further consists of, a nanotube-growth inhibit layer such as Si and Si₃N₄. In one aspect, the catalyst is any one or more of a particulate carbon-nanotube-growth-promoting catalyst or an iron- or cobalt-containing catalyst, or a mixture thereof. In a further aspect, the catalyst-particles on the substantially perpendicular walls are at a concentration greater than about 100 catalyst-particles per square micrometer.

Accordingly, in accordance with the above description, this invention also provides oriented carbon-nanotube-containing-solid comprising, or alternatively consisting essentially of, or yet further consisting of, the solid support system comprising, or alternatively consisting essentially of, or yet further consisting of, a plurality of carbon nanotubes grown in situ on the particles of the carbon-nanotube-growth-promoting catalyst. The plurality of carbon nanotubes are grown in situ on the particles of the carbon-nanotube-promoting catalyst present in the layer of catalyst on the substantially perpendicular wall of the indentations. In one aspect, the at least a portion of the carbon nanotubes bridge the substantially perpendicular walls of an indentation. The density of the nanotubes can be determined by modifying the parameters of catalyst and oxide layer and one of skill in the art can achieve a support having suspended nanotubes at a concentration of about 10 to about 100 carbon nanotubes per square micrometer. As noted above, one or more identical or non-identical receptors as described herein can be attached the nanotubes using methods known in the art and described herein.

The nanotube/receptor structures of this invention are useful to detect the presence of an analyte in a sample. To that end, this invention also provides a method of detecting analyte in a sample by contacting a sample suspected of containing the analyte with one or more of the sensor structures described above and herein under suitable conditions that favor binding of analyte to the receptor and detecting analyte bound to the receptor.

For the purpose of illustration only, the sample is any one or more of a liquid, gas, or plasma composition or biological sample such as blood; urine; cerebral spinal fluid; or microbial, viral or cellular sample. When the sample comprises, or alternatively consists essentially of, or yet further consists of gas, the gas can be contacted at about atmospheric pressure.

Binding of any analyte in the sample can be detected by measuring change in atomic vibration of the nanotube(s) which can be accomplished by methods known to those of skill in the art such as Raman spectroscopy radial breathing mode or in-plane graphene vibration. In one aspect, when the receptor comprises a label such as a fluorescent molecule, detection of binding of analyte to receptor is by measuring change in fluorescence emission by fluorescent spectroscopy or alternatively by fluorescent spectroscopy and Raman spectroscopy in tandem.

To achieve the detection of any analyte in the sample, the invention provides a sensor as described above and a means for detecting an analyte bound to the receptor, the means being determined by the receptor, nanotube and analyte to be detected. For example, when a change in fluorescence is to be detected, the means to detect can be a filter fluorometer or a spectrofluorometer. These devices are commercially available. See the web page: materialhandling.globalspec.com/Industrial-Directory/fluorescent_spectroscopy (last accessed on Feb. 12, 2009) for a listing of devices and vendors. Examples of spectroscopy devices include but are not limited to UV-visible/near infrared, Fourier transform-infrared (FT-IR), Fourier transform-Raman, dispersive Raman and fluorescence spectrometers which can be purchased from JASCO Inc. (Easton, Md., USA), HORIBA Jobin Yvon Inc. (Edison, N.J., USA), Princeton Instruments (Trenton, N.J., USA) and Ocean Optics (Dunedin, Fla., USA).

The invention also provides a method for preparing the orientated carbon-nanotube-containing solid disclosed herein, which comprises, or alternatively consists essentially of, or yet further consists of the steps of a) depositing a solid oxidic layer on a solid support having an indentation; b) depositing a layer of catalyst-particle-containing polymer on the support and at least within the indentation; and c) producing a plurality of nanotubes wherein at least a portion of the nanotubes bridge the indentation. In certain embodiments, the solid support comprises, or alternatively consists essentially of, or yet further consists of a silicon wafer.

Also provided herein is a method for preparing the molecular sensor of the invention which comprises, or alternatively consists essentially of, or yet further consists of the steps of: a) depositing a solid oxidic layer on a solid support having an indentation; b) depositing a layer of catalyst-particle-containing polymer; and c) producing a plurality of nanotubes wherein at least one nanotube bridges the indentation; and d) attaching a receptor to the surface of the at least one nanotube bridging the indentation.

In each of the embodiments disclosed herein, the production of carbon nanotubes can be accomplished using a variety of methods, such as arc discharge, laser ablation and chemical vapor deposition. In certain embodiments, the producing a plurality of nanotubes comprises carbon vapor deposition.

In some embodiments, the indentation has a bottom surface substantially parallel to the surface of the solid support and wherein the bottom surface is not coated with the solid oxidic layer. This can be accomplished by etching the solid oxidic layer from the bottom surface of the indentation using methods such as anisotropic dry etching, for example SF₆ gas, prior to deposition of the catalyst layer. In another aspect, the indentation has a bottom surface substantially parallel to the surface of the solid support and comprises, or alternatively consists essentially of, or yet further consists of, a nanotube-growth inhibit layer such as Si and Si₃N₄.

Typically, the solid oxidic layer can be deposited using thermal methods. The catalyst-particle-containing polymer can then be deposited via spin-coating, for example. In one aspect, the catalyst-particle-containing polymer comprises, or alternatively consists essentially of, or yet further consists of any one or more of a particulate carbon-nanotube-growth-promoting catalyst or an iron- or cobalt-containing catalyst, or a mixture thereof. In a further aspect, the catalyst-particles on the substantially perpendicular walls of the indentation are at a concentration greater than about 100 catalyst-particles per square micrometer.

### BRIEF DESCRIPTION OF THE DRAWINGS

FIG. 1, panels a and b, show the Raman signals of suspended carbon nanotubes vs carbon nanotubes on a surface. a) The radial breathing mode is detectable only for suspended CNTs. b) The in-plane carbon oscillation mode (G band) has a much higher intensity for suspended CNTs.

FIG. 2, panels a through h, show the complete fabrication of the molecular sensor. a) A self-assembling catalyst-containing block copolymer is applied to a SiO₂ coated Si wafer. b) A patterned photoresist film is formed on top of the catalyst-containing block copolymer. c) The resist pattern is transferred to the catalyst-containing block copolymer by etching. d) The photoresist is removed by solvent stripping. e) The organic materials are removed by UV-ozonation, thus forming the catalyst-containing nanostructures. f) Suspended carbon nanotubes are then grown via chemical vapor deposition (CVD). g) After the suspended nanotubes are grown, they can be modified on the surface by the attachment of receptor molecules (i.e. antibodies). h) Exposure of the surface modified suspended CNTs to the appropriate analyte molecules would result in a binding event to the receptor.

FIG. 3 schematically illustrates detection of analyte bound to the molecular sensor via Raman spectroscopy. Analogously, a fluorimeter can be implemented wherein the detection of analyte bound to the molecular sensor is by fluorescence spectroscopy.

FIG. 4 shows a SEM image of suspended CNTs.

FIG. 5 shows alternative architectures for and the growth of suspended CNTs.

FIG. 6, panels a through c, show the fabrication of a 3-dimensional molecular sensor, including the synthesis of a solid support system. a) A photoresist is applied and patterned on a silica wafer. The pattern is then transferred to the silica wafer using wet etching techniques. Thermal oxide growth followed by anisotropic dry etching prepares the solid for the catalyst deposition, after which carbon nanotubes can be grown in situ. b) The solid support system with oriented suspended carbon nanotubes, wherein c) the indentations of the solid support system have substantially perpendicular walls.

FIG. 7, panels a and c, depict tilted SEM images (3 μm×2 μm filed of view) showing that suspended SWNTs aligned orthogonally to trench orientation. The optical image (panel b) shows that this set of trenches are in close proximity, oriented 90° with respect to each other. The arrows in the images indicate tube orientations that are approximately perpendicular to trench orientation.

### DETAILED DESCRIPTION OF THE INVENTION

Throughout this disclosure, various publications, patents and published patent specifications are referenced. The disclosures of these publications, patents and published patent specifications are hereby incorporated by reference into the present disclosure to more fully describe the state of the art to which this invention pertains.

### DEFINITIONS

As used herein, certain terms may have the following defined meanings.

As used in the specification and claims, the singular form “a,” “an” and “the” include singular and plural references unless the context clearly dictates otherwise. For example, the term “a carbon nanotube” includes a single carbon nanotube and as well as a plurality of carbon nanotubes.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps for preparing the molecular sensor. Embodiments defined by each of these transition terms are within the scope of this invention. Accordingly, it is intended that the methods and compositions can include additional steps and components (comprising) or alternatively including the steps and compositions of no significance (consisting essentially of) or alternatively, intending only the stated methods steps or compositions (consisting of).

As used herein the term “nanotube” is intend to mean a cylindrical tubular structure of which the most inner diameter size lies between 0.5 nm and 1000 nm. A nanotube is a member of the fullerene structural family, which also includes buckyballs. Whereas buckyballs are spherical in shape, a nanotube is cylindrical, with at least one end typically capped with a hemisphere of the buckyball structure. Nanotubes are composed primarily or entirely of sp² bonds, similar to those of graphite. This bonding structure, stronger than the sp^{a} bonds found in diamond, provides the molecules with their unique strength.

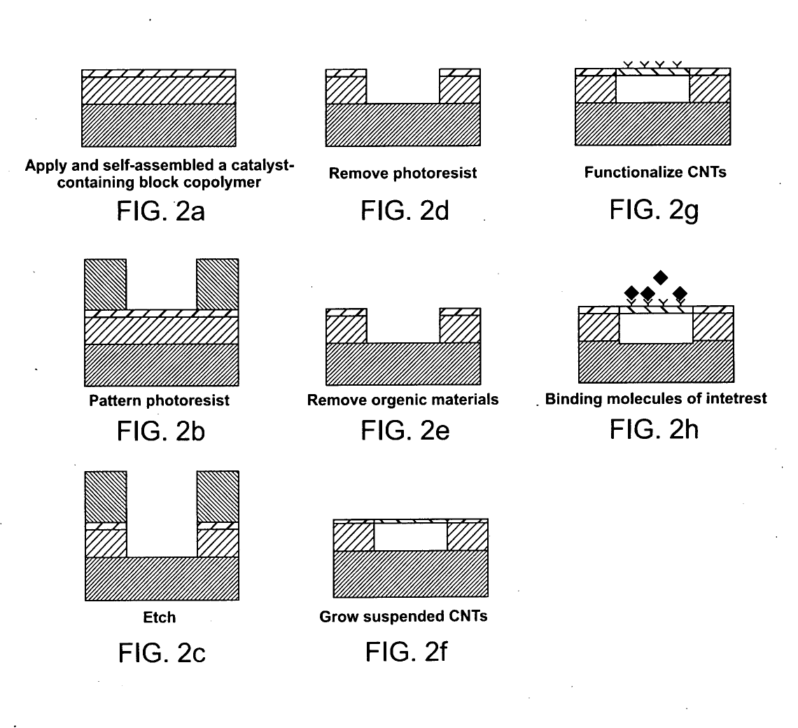

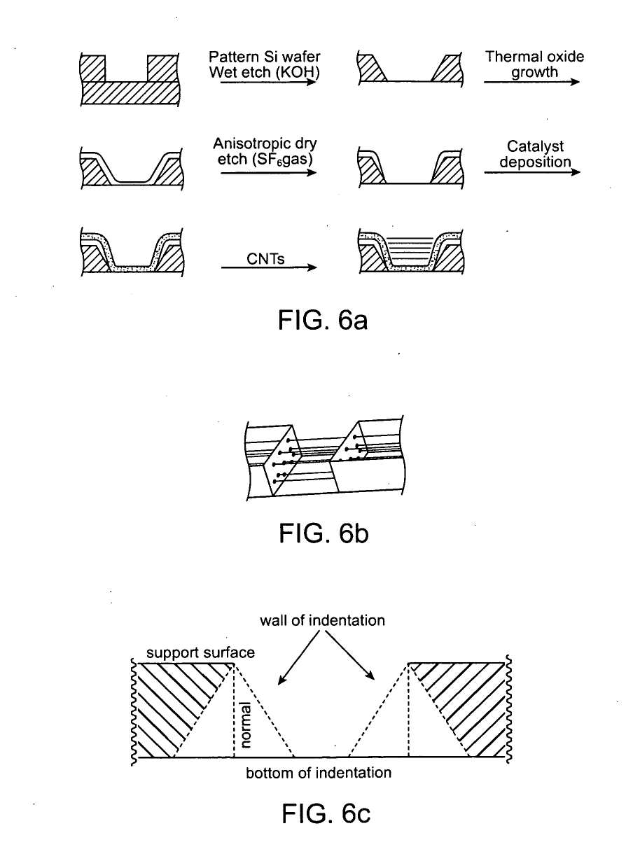

As used herein the term “suspended carbon nanotube” is intend to mean a carbon nanotube suspended across an indentation of a solid support. The term “solid support” is intend to mean a catalyst-containing template as shown in FIG. 2e and FIG. 6a, on which suspended CNTs can be grown. The terms “catalyst” and “catalyst-particle” are used interchangeably and are intended to refer to a carbon-nanotube-growth-promoting or nucleating-catalyst, such as those containing iorn, cobalt, or any other suitable carbon-nanotube-growth-promoting or nucleating-catalyst or mixture thereof. The size of the catalyst-particles on the “solid support” control the diameter of the suspended carbon nanotubes. The synthesis of suspended carbon nanotubes on such support structures that can be used in the present invention include, but are not limited to those which are known in the art (Lu, et al. (2006) J. Phys. Chem. B. 110: 10585-10589; Cassell, et al. (1999) J. Am. Chem. Soc. 121: 7975-7976). The suspended nanotubes synthesized on the support structures of the disclosed invention are to be differentiated from the vertically aligned or bundled nanotubes known in the art (Zhao, et al. US Pub. No. US 2006/0252065, filed Mar. 5, 2006).

The term “substantially perpendicular” when referring to the walls of the indentations of the solid support, is intended to refer to the relationship, or angle, between the wall of the indentation and the surface of the support surface. “Substantially perpendicular” can encompass angles which differ from the normal by as much as 45°. This is shown schematically in FIG. 6c. Acceptable angles range from about 45 to about 90°.

Likewise, the term “substantially parallel” when referring to the bottom of the indentations of the solid support, is intended to refer to the respective angle between the bottom surface of the indentation and the support surface. “Substantially parallel” surfaces of the disclosed invention can have surfaces where the plane angles are from within about 25° of parallel, or alternatively, about 20°, or alternatively, about 15°, or alternatively, about 10°, or alternatively, about 5°, or alternatively, about 0°. The bottom of the indentation can also be rounded, or have rounded corners.

The “3-dimensional suspended carbon nanotube network” of the current invention is as shown in FIG. 6. Using the 3-dimensional catalyst containing nanostructure, an array of suspended carbon nanotubes can be grown to span the walls of the 3-dimensional catalyst containing nanostructure. The multiple layers of suspended nanotubes can be used to enhance the signal for the detection of analyte.

The term “oriented” is intended to refer to a suspended carbon nanotube or a series of carbon nanotubes that grow outwardly toward an adjacent surface or wall wherein the nanotube will be suspended across an indentation. Whereas a well shaped indentation would generate a high population of suspended CNTs with a lot of crossovers, a trench shape indentation may result in less crossover.

As used herein, the term “surface-modified” is intend to mean modifying any surface of a suspended carbon nanotube with a receptor, and includes, but is not limited to, more than a simple application or layering on the surface. For example, surface modification may comprise receptor attachment to the surface of the suspended CNT by Van der Waals forces, ionic bonds, covalent bonds, hydrogen bonds, or any other chemical bonds or methods. The modifications may or may not be permanent and in some cases may be reversible. The CNTs can also optionally be chemically modified with synthetic handles, such as carboxylic acid, ester, hydroxyl or amine functional groups to enable a covalent bond to the receptor. Methods for the modification of carbon nanotubes and nanowires, including attachment of a receptor, are known (Lieber, et al. U.S. Pat. No. 7,256,466; Chen, et al. (2003) PNAS 100:9 4984-4989; Lin, et al. (2004) J. Mater. Chem. 14: 527-541; McCall, et al. Pub. No. US 2006/0246438 A1; Boussaad, et al. Pub. No. US 2006/0194263 A1).

The CNTs can be treated with a desired biomarker (sensing element or receptor). Any biomarker can potentially be used as long as the space between the adjacent CNTs is sufficient to allow analyte binding. The term “biomarker” or “receptor” refers to any species which specifically binds to an analyte so as to allow the measurement of the presence or quantity of the analyte. Receptors which recognize a particular analyte are said to be complementary to that analyte. Suitable receptors include, by way of example, antibodies, antibody fragments, antigens, haptens, nucleic acids, particularly single stranded nucleic acids, cells, hormones, binding proteins, oligosaccharides, lectins, avidin, biotin, protein A, protein G, and the like. A receptor and its complementary analyte are sometimes referred to as a receptor/analyte binding pair. Receptor/analyte binding pairs are well known in the art and include antigen/antibody pairs, biotin/avidin pairs, lectin/oligosaccharide pairs, single stranded nucleic acids and a complementary single or double stranded pair, and the like. The presence or amount of analyte in a sample is determined by virtue of binding the analyte present in the sample to the “surface-bound receptor,” e.g., an antibody in the case of an antigen analyte. The term “surface bound receptor” is intended to mean a molecule or ligand immobilized on the surface of a suspended carbon nanotube.

As used herein the term “analyte” is intend to mean a molecule of interest that is to be analyzed and can be any molecule or compound. The analyte could be an organic or inorganic molecule. Non-limiting illustrative examples of analytes may include a small molecule, biomolecule, or nanomaterial such as but not necessarily limited to a small molecule that is biologically active, nucleic acids and their sequences, peptides and polypeptides and biomolecules or small molecules capable of binding to molecular probes on chemically modified carbon nanotubes. The analyte molecule may be DNA or RNA. An analyte can be in the solid, liquid, plasma, gaseous or vapor phase. The term “analyte” is also intended to encompass molecular species which are functionally equivalent to analytes of interest in a particular context.

The term “ligand” refers to a biological or chemical molecule that is able to bind to and form a complex with a biomolecule to serve a biological purpose. In one embodiment, a ligand is an effector molecule binding to a site on a target protein, by intermolecular forces such as ionic bonds, hydrogen bonds and Van der Waals forces. This association is typically reversible. Antibodies and enzymes are non-limiting examples of ligands.

The terms “binding” and “bound” as used herein are meant to include interactions between molecules such as an analyte and receptor or alternatively ligand and antibody. Interactions may be, for example, protein-protein, protein-nucleic acid, protein-small molecule or small molecule-nucleic acid in nature. This binding can result in the formation of a “complex” comprising the interacting molecules. A “complex” refers to the binding of two or more molecules held together by covalent or non-covalent bonds, interactions or forces. A “binding partner” is intended to mean a specific binding substance that specifically binds to its binding partner, but does not substantially bind other binding partners added to the surface of a biosensor. For example, where the specific binding substance is an antibody and its binding partner is a particular antigen, the antibody specifically binds to the particular antigen, but does not substantially bind other antigens. A binding partner can be, for example, a nucleic acid, polypeptide, antigen, polyclonal antibody, monoclonal antibody, single chain antibody, small organic molecule, cell, virus and bacteria.

The term “Raman spectroscopy” as used herein is intended to mean a spectroscopic technique used to study vibrational, rotational, and other low-frequency modes in a system. Examples of “Raman sensitive” atomic vibration modes include, without limitation, radial breathing mode, in-plane graphene vibration and second-order harmonic modes. It relies on inelastic scattering, or Raman scattering of monochromatic light, usually from a laser in the visible, near infrared, or near ultraviolet range. Other methods for detecting molecular vibration include, but are not limited to, Fourier transform Raman spectroscopy, hyper-resonance Raman spectroscopy (Wang, et al. (2005) Science, 308: 838-841; Kneipp, et al. (2006) PNAS, 103:46 17149-17153), infrared spectroscopy and Fourier transform infrared spectroscopy.

The term “fluorescent” refers to any substance or agent that is capable of exhibiting “fluorescence” (or “photoluminescence”), which is the emission of light triggered by the molecular absorption of a photon with longer wavelength. Fluorescence thus is dependent on an “excitation light source” that is distinct from the longer wavelength fluorescence “emission” emanating from the fluorophore. Detection of fluorescence emission requires that a detector that responds only to the emission light and not to the excitation light. As is known to those of skill in the art, the detectable response is a change in a property of the luminescence or fluorescence light, such as a change in the intensity, polarization, energy transfer, lifetime, and/or excitation or emission wavelength distribution. The detectable response may be simply detected, or it may be quantified. A response that is simply detected generally comprises a response whose existence merely is confirmed, whereas a response that is quantified generally comprises a response having a quantifiable (e.g., numerically reportable) value such as an intensity, polarization, and/or other property.

As used herein, the term “transducer” is intend to mean a species that converts a signal from one form to another to allow for signal detection. For example, an “environmental perturbation” such as a binding event or change in temperature, pressure, molecular vibration, shape or current, causes a detectable signal change in the transducer. As used herein, the term “environmental perturbation” is intend to mean a variable change in the local environment of the transducer, such as the occurrence of a binding event upon contact with a sample.

As used herein, the term “sample” is intend to mean a solution, gas, liquid, plasma or any other medium, biological or inorganic, suspected of containing a molecule or analyte that can be evaluated in accordance with the invention.

The following abbreviations are used throughout this disclosure.
AFM=atomic force microscopy/atomic force microscope
CNT=carbon nanotube
CVD=chemical vapor deposition
ICP=inductively coupled plasma
SEM=scanning electron microscope
IR=infrared spectroscopy
MWNT=multi walled nanotube
SWNT=single walled nanotube
μm=micrometer
nm=nanometer
min=minute
m=meter
s=second
° C.=degrees Celsius
sccm=Standard Cubic Centimeters per Minute, where “Standard” means referenced to 0° C. and 760 Torr.
rpm=revolutions per minute
PS-b-P2VP=polystyrene-b-poly(2-vinylpyridine)
PS-b-PFEMS=polystyrene-b-polyferrocenylethylmethylsilane

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

### EMBODIMENTS

The molecular sensor disclosed herein utilizes suspended CNTs with a diameter of less than about 10 nm and can be as small as 0.5 nm. For Raman detection of the radial breathing mode, CNTs with a diameter of less than about 2 nm are required. CNT diameter can be controlled by controlling the particle size of the catalyst on which the CNT is fabricated. A 3D CNT configuration in FIG. 6 will increase the number of CNTs in a given area and thus enhance the detection signal. The CNTs can then be treated with a desired biomarker (sensing element). Any biomarker can potentially be used as long as the space between the adjacent CNTs is sufficient to allow analyte binding. Covalent or non-covalent methods can be used to attach the sensing element to the CNT. For covalent methods, the CNTs can be functionalized with handles (hydroxyl or carboxyl moieties, for example) with which to bond the sensing element. Alternatively, the sensing element can be attached to the CNT by non-covalent methods. The specificity of the molecular sensor is determined by the specificity of the given biomarker. The sensitivity of the molecular sensor is hypothesized to be at the molecular level. This novel molecular sensor may be used to identify and/or sense the presence, concentration, or absence of various molecules or analytes in a liquid, gas, or plasma state.

### Molecular Sensors Comprising Suspended Carbon Nanotubes and Methods for Preparing the Same

In one aspect, the present invention discloses a molecular sensor comprising at least one carbon nanotube suspended on a suitable support structure having at least one receptor attached to a surface of said suspended carbon nanotube. Alternatively, the present invention discloses a molecular sensor comprising a plurality of molecular sensors positioned proximate to each other to allow binding of at least one receptor to its binding partner thereby forming a 3-dimensional network and improving the detection capability of the sensor. A molecular sensor comprising a 3-dimensional suspended carbon nanotube network, thereby improving the detection capability of said sensor.

In a particular embodiment, the present invention discloses a molecular sensor comprising at least one carbon nanotube suspended on a suitable support structure having a plurality of receptors attached to a surface of said suspended carbon nanotube. Further, this can incorporate an plurality of the CNT-based molecular sensors disclosed herein. Each suspended nanotube or molecular sensor can be functionalized with a specific biomarker. Therefore, arrays of sensors can allow for the detection of multiple receptors simultaneously. This is also known as multiplex detection.

In an alternative aspect, the present invention discloses a molecular sensor comprising an oriented carbon-nanotube-containing-solid, wherein a plurality of carbon nanotubes are grown in situ on the particles of a particulate carbon-nanotube-growth-promoting catalyst, wherein at least one receptor is attached to a surface of said carbon nanotubes. Further, the oriented carbon-nanotube-containing-solid of the above described molecular sensor includes a plurality of carbon nanotubes grown in situ on the particles of the carbon-nanotube-promoting catalyst present in the layer of catalyst on the substantially perpendicular wall of the indentations, wherein at least a portion of the carbon nanotubes bridge the substantially perpendicular walls of an indentation.

In the particular embodiment wherein the method of detection uses the radial breathing mode, at least one of the suspended carbon nanotubes of the above disclosed molecular sensors has a diameter less than about 2 nm. Alternatively, said suspended carbon nanotube has a diameter less than about 1.8 nm, and alternatively, less than about 1.5 nm, and alternatively, less than about 1.3 nm, and alternatively, less than about 1.0 nm, and alternatively, less than about 0.75 nm, and alternatively, about 0.5 nm. Alternatively, if the suspended nanotubes have a diameter larger than 2 nm, the analyte can be detected using Raman spectroscopy via the G-band.

In the above molecular sensors, the receptor can be attached to the surface of a suspended carbon nanotube. One or more of any suitable bonding methods can be used, such as Van der Waals forces, ionic bonds, covalent bonds, hydrogen bonds or any other chemical bonds or methods. Non-limiting examples of the various receptors that can be utilized in the present invention include antibodies, antibody fragments, antigens, haptens, nucleic acids, particularly single stranded nucleic acids, cells, hormones, binding proteins, oligosaccharides, lectins, avidin, biotin, protein A, protein G, and the like. In addition, the receptor can be a ligand, referring to a biological or chemical molecule that is able to bind to and forms a complex with a biomolecule to serve a biological purpose. In one embodiment, a ligand is an effector molecule binding to a site on a target protein, by intermolecular forces such as ionic bonds, hydrogen bonds and Van der Waals forces. This association is typically, although not required to be, reversible. Antibodies and enzymes are non-limiting examples of ligands.

Methods for preparing molecular sensors comprising suspended carbon nanotubes are described herein. FIG. 2 shows the complete fabrication of a molecular sensor which is encompassed by the disclosed invention. Central to the disclosed invention is the ability to generate suspended single walled carbon nanotubes (SWNTs). Suspended SWNTs can be generated via chemical vapor deposition using a catalyst-containing support structure.

The support structure for the controlled growth of suspended SWNTs is constructed from a self-assembling catalyst-containing block copolymer which is applied to a Si/SiO₂ wafer (FIG. 2a) For the growth of suspended nanotubes, a series of indentations are etched into both the block-copolymer layer and the SiO₂ underlayer using a patterned high contrast photoresist (FIGS. 2b and 2c). Other known photoresists can be used to make the support structures disclosed herein, such as high contrast or low contrast broadband photoresists. After stripping the photoresist, UV-ozonation converts the catalyst-containing polymer into either cobalt oxide nanoparticles or iron-containing silica nanostructures thus preparing the catalyst for nanotube growth (FIGS. 2d and 2e). Nanotubes growing toward adjacent towers or ledges of the support structure become suspended. The CNTs which fall into the trenches are not easily resolved. Various structural designs can be employed for the synthesis of suspended of the nanotubes. In addition, the use of a multi-level 3-dimensional support structure is included within the scope of the invention for the synthesis of multiple layers of suspended nanotubes.

Alternatively, a particular embodiment of the present invention discloses a solid support having an indentation with walls substantially perpendicular to the surface of the solid support, the substantially perpendicular walls being coated with a solid oxidic layer and over-coated with a layer of a catalyst-particle-containing polymer. Such a structure can be fabricated as depicted in FIG. 6. In one embodiment, the solid support is a nanoscale solid support. In a particular embodiment, the disclosed invention describes a solid inorganic support, or alternatively, a nanoscale solid inorganic support. A silica wafer, for example, can be patterned using a photoresist and standard wet etching techniques to form at least one indentation. In alternative embodiments of the present invention, the indentation can be a trench, well, or any other suitable pattern provided the wall of the indentation is substantially perpendicular. Wet etching is preferred as in order to grow suspended CNTs wherein they grow from one wall of the indentation and land on the opposite site, sloped side walls are desirable. In particular embodiments, the respective angle between the wall of the indentation and the support surface is greater than about 45°, or alternatively, about 55°, or alternatively, about 60°, or alternatively, about 70°, or alternatively, about 80°, or alternatively, about 90°. Alternatively, the respective angle between the wall of the indentation and the support surface can be greater than about 90°, provided at least one suspended nanotube can be grown on the solid support.

After removal of the photoresist, the etched solid support is coated with a solid oxide layer which can be grown by thermal deposition methods. In a particular embodiment, the indentations of the solid support system have bottom surfaces in which the bottom surfaces are not coated with the solid oxidic layer. This can be accomplished by anisotropically etching the thermal oxide growth using known methods such as SF₆ gas (FIG. 6a). Other known anisotropic dry etching methods, such as HF or other fluorine or chlorine containing gas, can be used.

In an alternative embodiment, the present invention discloses a solid support system wherein the indentations have bottom surfaces which comprise a growth inhibit layer, such as Si and Si₃N₄.

In a particular embodiment, the catalyst is a particulate carbon-nanotube-growth-promoting-catalyst. As discussed herein, in order to control the diameter of the CNTs in general, one must use uniformly sized catalyst particles. In addition, the nanotube growth sites can be controlled by careful deposition of the catalyst particles at predefined locations. The deposition of the catalyst-particles can be accomplished by over-coating the solid support system via spin coating a solution of polymeric catalyst precursor materials such as iron complexed PS-b-P2VP micellar solution. In a particular embodiment, the carbon nanotube growth promoting catalyst-particles can be iron- or cobalt-containing particles, or a mixture thereof.

In one aspect, the present invention discloses an oriented carbon-nanotube-containing-solid with a plurality of carbon nanotubes bridging the pattern of indentations in the solid support, the nanotubes being grown in situ between particles of the carbon nanotube promoting catalyst present in the layer of catalyst on the substantially perpendicular wall of the indentations. In a particular embodiment of the present invention, at least a portion of the carbon nanotubes bridge an indentation, forming suspended carbon-nanotubes.

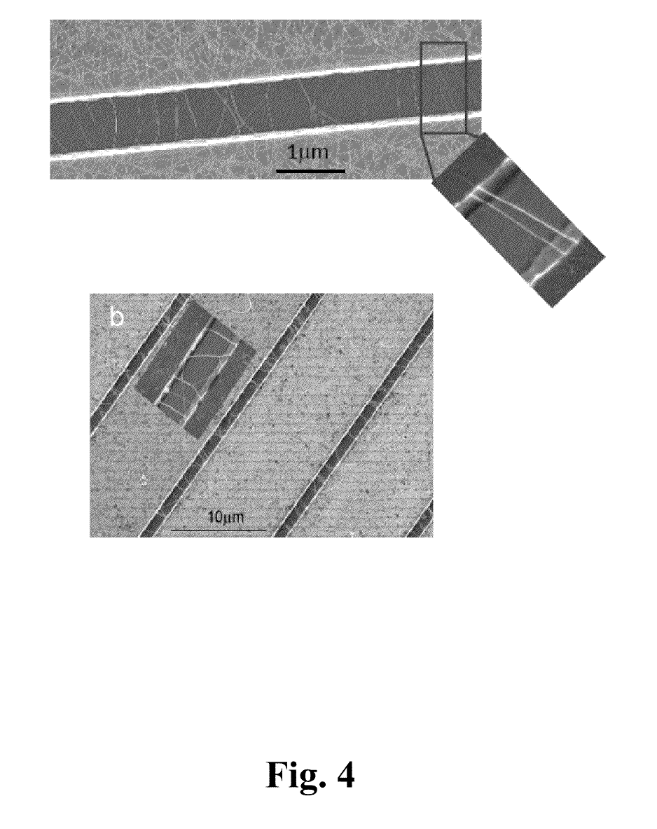

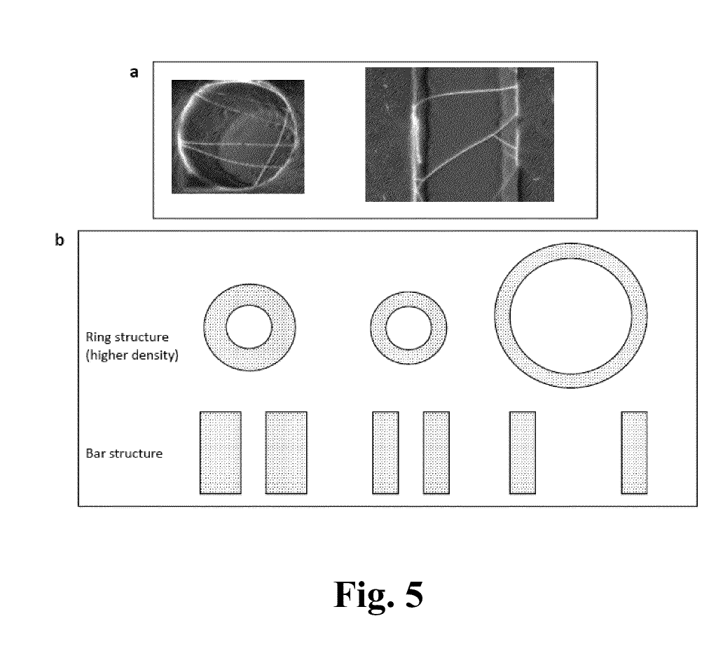

For the synthesis of suspended CNTs in general, for all of the molecular sensors disclosed herein, the etching of the solid support should provide at least one indentation where the substantially perpendicular walls are separated by a distance from about 0.3 to about 10 μm or alternatively, towers from about 0.3 to about 10 μm apart. In a particular embodiment, the etching provides at least one indentation where the substantially perpendicular walls are separated by a distance of about 1 μm (FIG. 4), or alternatively less than about 1 μm, or alternatively about 2 μm, or alternatively about 3 μm, or alternatively about 4 μm, or alternatively about 5 μm, or alternatively about 6 μm, or alternatively about 7 μm, or alternatively about 8 μm, or alternatively about 9 μm, or alternatively about 10 μm. In an alternative embodiment, the etching process provides wells wherein the suspended CNTs are grown across the well or ring as shown in FIG. 5.

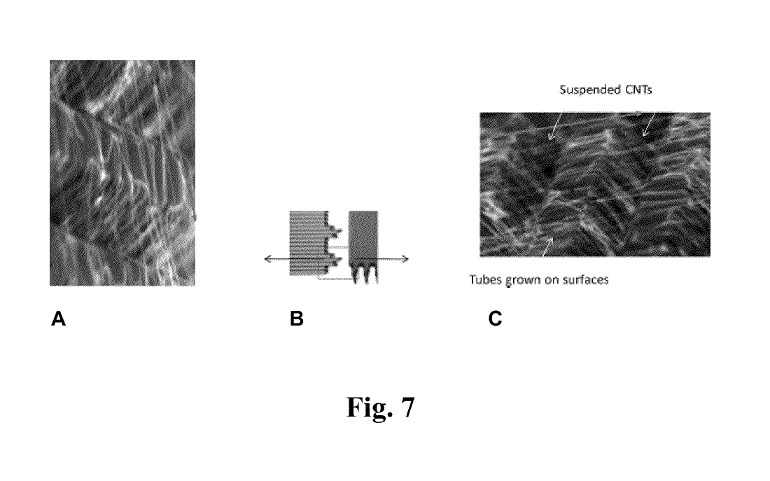

Carbon nanotube orientation can be controlled by surface topography. As shown in the optical image in FIG. 7, suspended CNTs have been synthesized from patterned surfaces with neighboring trenches oriented perpendicularly. The tilted SEM images are 3D CNTs grown in these trenches. It can be seen that suspended CNTs are oriented orthogonally to the sidewalls of trenches and substantially parallel with each other. CNTs could grow randomly in any direction without discretion. A small portion of tubes grow on plateaus as can be seen clearly from the SEM images in FIG. 5. However, only tubes with proper orientation can grow across a trench. The shorter the distance is, the more likely it is that CNTs will grow from one sidewall and reach the opposite sidewall surface. During CNT synthesis, their ends grow until they touch the opposite sidewall. CNTs with their orientation perpendicular to a trench have the shortest suspension length or travel distance, therefore are more likely to occur. This result suggests that surface topography can be employed to control tube orientation. By combining this effect with other methods such as carbon precursor gas flow direction, it is contemplated that CNT orientation can be further improved.

The concentration of suspended the suspended carbon nanotubes is dictated by the concentration of catalyst-particles on the support structure. The concentration of catalyst particles can be varied to achieve the desired concentration of suspended CNTs. In a particular embodiment, for the carbon nanotubes grown on the substantially perpendicular walls of the solid support structure disclosed herein, the concentration of catalyst-particles is greater than about 100 catalyst-particles per square micrometer. In an alternative embodiment, the concentration of catalyst-particles is less than about 100 catalyst-particles per square micrometer, or alternatively, less than about 90, or alternatively, less than about 80, or alternatively, less than about 60, or alternatively, less than about 50, or alternatively, less than about 40, or alternatively, less than about 25. However, the yield of suspended carbon nanotubes can vary, resulting in a concentration range of the suspended carbon nanotubes.

In a particular embodiment, the suspended carbon nanotubes on the oriented carbon nanotube containing solid which bridge an indentation of the solid support system, would be at a concentration of about 10 to about 100 per square micrometer. In an alternative embodiment, the suspended carbon nanotubes on the oriented carbon nanotube containing solid which bridge an indentation of the solid support system, would be at a concentration of about 10 to about 25 per square micrometer, or alternatively, about 25 to about 50, or alternatively, about 50 to about 75, or alternatively, about 75 to about 100, or alternatively, about 10 to about 50, or alternatively, about 10 to about 75, or alternatively, about 25 to about 75, or alternatively, about 25 to about 100, or alternatively, about 75 to about 100.

Carbon nanotubes are then grown via chemical vapor deposition (CVD) such that the carbon nanotubes span across the preformed trenches of the support structure and are thus suspended in air (FIG. 2f). One aspect of the invention disclosed herein describes sensing based on lattice vibration signals. Therefore both metallic and semi-conducting CNTs can be used. Other synthesis defects such as amorphous carbon and defects can also be tolerated to some extent. After the suspended nanotubes are grown, they can be surface-modified by the attachment of receptor molecules (FIG. 2e).

The diameter of the suspended SWNTs is controlled by the size of the catalyst particles. With current Raman-sensing methods, the diameter of the SWNTs must be <2 nm to be detected via RBM, whereas SWNTs nanotubes having a diameter greater than 2 nm can be detected using Raman spectroscopy via the G-band. Only semiconducting CNT fluorescent. Therefore the detected fluorescence signals are emitted from semiconducting CNTs. The presence of metallic tubes will neither interfere nor quench the signals as long as they are not bundled with semiconducting CNTs.

### Methods for the Detection of Analytes Using the Molecular Sensor

Once constructed, the molecular sensors described herein are capable of binding various analyte molecules by contacting the molecular sensor with a sample under suitable conditions that favor binding of the analyte to the receptor. Disclosed herein is a method of detecting analyte in a sample, comprising contacting the sample with the molecular sensors described hereinabove, under suitable conditions that favor binding of analyte to the receptor and detecting analyte bound to the receptor.

Alternatively, the present invention is directed to a system for detecting the presence of an analyte in a sample comprising the molecular sensors described hereinabove, and a means for detecting an analyte bound to the receptor. In a particular embodiment, the present invention is directed to a method of detecting an analyte in a sample, comprising contacting the sample with the system under suitable conditions that favor binding of the analyte to the receptor and detecting any analyte bound to the receptor.

Non-limiting illustrative examples of analytes may include a small molecule, biomolecule, or nanomaterial such as but not necessarily limited to a small molecule that is biologically active, nucleic acids and their sequences, peptides and polypeptides and biomolecules or small molecules capable of binding to molecular probes on chemically modified carbon nanotubes. The analyte molecule may be DNA or RNA. The term “analyte” is also intended to encompass molecular species which are functionally equivalent to analytes of interest in a particular context.

In addition to the molecular sensor as shown in FIG. 2g, the present invention discloses a 3-dimensional suspended carbon nanotube sensor comprising a plurality of molecular sensors comprising at least one carbon nanotube suspended on a suitable support structure having at least one receptor attached to a surface of said suspended carbon nanotube positioned proximate to each other to allow binding of at least one receptor to its binding partner. An exemplary embodiment is shown in FIG. 6.

A binding partner can be, for example, a nucleic acid, polypeptide, antigen, polyclonal antibody, monoclonal antibody, single chain antibody, small organic molecule, cell, virus and bacteria. The specificity of the molecular sensor is therefore dependent on the interaction between the receptor and its binding partner. For example, where the specific binding substance is an antibody and its binding partner is a particular antigen, the antibody specifically binds to the particular antigen, but does not substantially bind other antigens.

One feature inherent to the molecular sensors disclosed herein is the lower noise measurement. Typically, an individual CNT or CNT mat is grown on SiO₂ and thus attaching receptors for target molecules on CNTs may also attach them on the SiO₂ due to nonspecific binding. Both Raman and fluorescence signals of CNTs attached on a surface will be quenched. This nonspecific binding will contribute to measurement noise if electronic-based approach is used for sensing. Since the described method only sense signals from suspended CNTs, non-specific binding does not add any noise to the measurement.

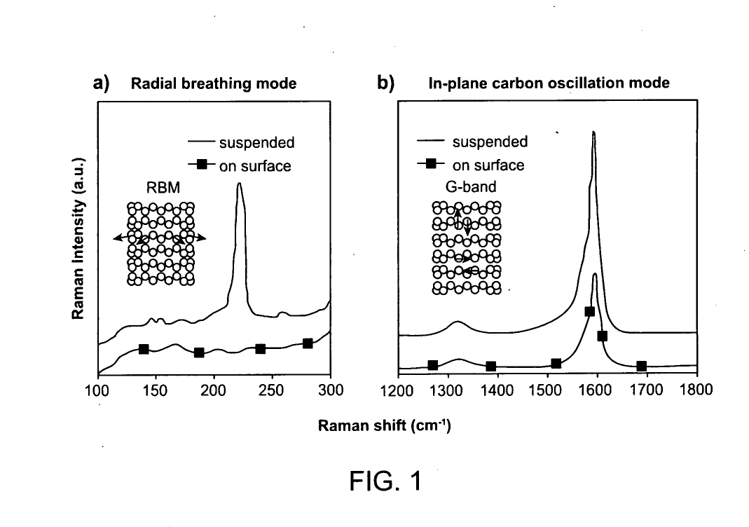

Raman signals contain structurally sensitive information and thus can be used for highly sensitive chemical detection. A CNT contains several extremely Raman-sensitive modes including the radial breathing mode (RBM), the in-plane sp² atomic vibrational mode (G-band) and the second-order harmonic mode. The signals of these modes can be greatly enhanced by suspending the CNTs in air, free of Van der Waals interaction. Biomolecule binding will cause changes in electronic and vibrational states of a CNT (FIG. 1, (J. Lu, et al. (2006) J. Phys. Chem. B, 110:22, 10585-10589). Therefore detection of change in Raman signals of an individual CNT or a group of CNTs can be used for ultra-sensitive sensing.

Exposure of the surface modified suspended CNTs to the appropriate analyte molecules would result in a binding event to the receptor (FIG. 2h). The sample, can be in the liquid, gas, or plasma state. An example of a sample includes, but is not limited to, a biological sample, wherein the analyte is a nucleic acid, polypeptide, antigen, polyclonal antibody, monoclonal antibody, single chain antibody, small organic molecule, cell, virus or bacteria. The gas can be at various pressures, including atmospheric pressure. Therefore, the analyte can either be a gaseous analyte, or an analyte in the vapor phase.

The present invention discloses a system for detecting the presence of an analyte in a sample comprising any of the above described molecular sensors comprising at least one carbon nanotube suspended on a suitable support structure having at least one receptor attached to a surface of said suspended carbon nanotube and a means for detecting analyte bound to the receptor. The analyte receptor binding event can be detected using a variety of spectroscopic methods. Non-limiting examples include Fourier transform Raman spectroscopy, hyper-resonance Raman spectroscopy, infrared spectroscopy and Fourier transform infrared spectroscopy.

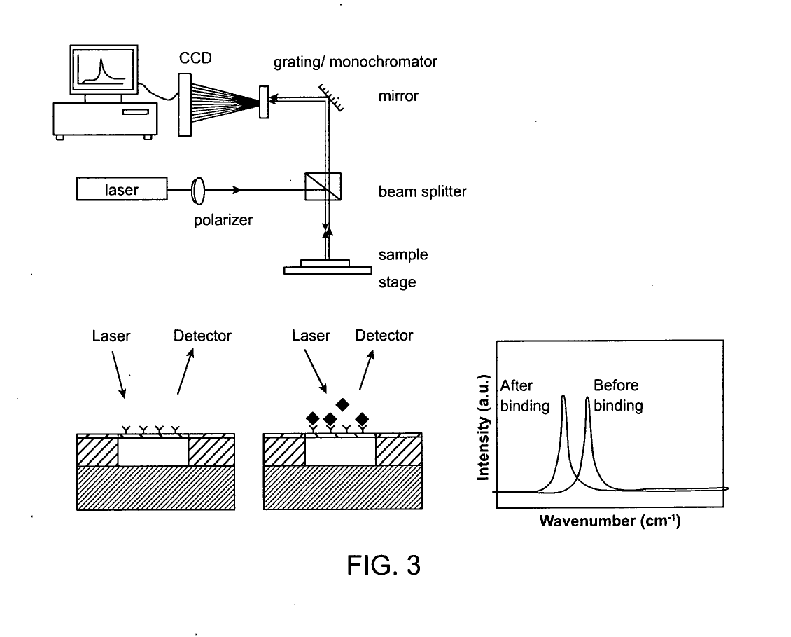

Schematically, as illustrated in FIG. 3, detecting of binding of analyte to receptor is by measuring change in atomic vibration, wherein the measured change in atomic vibration is radial breathing mode or in-plane graphene vibration. The change in atomic vibration is measured by Raman spectroscopy. In a particular aspect, the present invention discloses a molecular sensor comprising at least one surface-modified suspended carbon nanotube, wherein contact of an analyte to the surface bound receptor changes the carbon nanotube vibrational properties, and the shift of vibration modes can be detected by Raman spectroscopy.

In a particular embodiment, the present invention is directed to a molecular sensor comprising at least one suspended carbon nanotube as a transducer, based on measuring change (in intensity or shift), of atomic vibration of Raman sensitive modes upon environmental perturbation, wherein a receptor is first attached to the surface of said carbon nanotube. The vibrational modes can be radial breathing mode (RBM) and/or in-plane graphene vibration (G-band). Qualitative and quantitative detection of biomolecule attachment can be accomplished by measuring the changes of Raman lattice vibrational modes before and after biomolecule binding. Current CNT synthesis methods yield a mixture of metallic and semiconducting tubes. This imposes the major hurdle for realization of the electrical-based detection. Raman can be used to detect changes for both metallic and semiconducting tubes before and after binding.

Alternatively, detection of the binding of the analyte to the receptor can be accomplished by measuring change in fluorescence emission. Carbon nanotubes are known to absorb and give off light in the near-infrared spectrum. However, the fluorescence of the CNT is quenched if the nanotubes are bundled or attached onto a surface of the support structure. The molecular sensor disclosed herein comprises individual surface-modified suspended CNTs and thus would permit analyte detection by measuring change (in intensity or shift) fluorescence emission by fluorescence spectroscopy. Accordingly, in one aspect of the present invention, the detection of an analyte binding to a receptor is by measuring delta fluorescence emission using fluorescence spectroscopy.

In addition to discrete sensing using either Raman spectroscopy or fluorescence spectroscopy, the sensing methods can be utilized in tandem. This would allow for a simple measurement verification system in order to assure measurements with a low error.

The above described molecular sensor uses a simple architecture with great manufacturability. Moreover, biosensing based on this optical means can be easily integrated into device applications such as lab-on chips. Combining the extremely large surface area with exceedingly sensitive properties to environmental changes make a CNT very promising as a sensing element. In a particular embodiment, the present invention discloses a molecular sensor comprising a 3-dimensional suspended carbon nanotube network, thereby improving the detection capability of the sensor. The lab-on chips can be incorporated into a variety of devises including, but not limited to, microfluidic devices and flow-thru devises, and as such, can be used with any of the detection methods described herein. For the flow-through devises, the molecular sensor described herein can be mounted in an appropriate housing, such as a quartz cuvette, equipped with openings on each side for sample circulation (flow-through). Such apparatus' are known and can be purchased from commercial sources (Shimadzu Scientific Instruments, www.ssi.shimadzu.com). For liquid samples, the flow-through housing can be in line with a peristaltic pump for sample circulation.

This method fully utilizes the highly sensitive properties of CNTs, and at the same time can tolerate CNT synthesis imperfection which hinders all other proposed CNT sensing methods from commercialization. This method described herein greatly alleviates the stringent synthesis requirements imposed by conventional electronic-based sensing. Current CNT synthesis methods yield a mixture of metallic and semiconducting tubes. This imposes the major hurdle for realization of the electrical-based detection. Raman can be used to detect changes for both metallic and semiconducting tubes before and after binding.

### Example 1

### Generation of Suspended CNTs

A catalyst-containing diblock copolymer solution was first spin-coated onto a Si wafer with a 500 nm thick thermally grown silicon oxide film. Upon spin-coating of cobalt-complexed PS-b-P2VP, a monolayer of highly ordered cobalt-loaded surface micelles was directly formed. After coating the PS-b-PFEMS solution, solvent annealing in toluene was then performed to promote the self-assembly process. Once the highly ordered cobalt- and iron-containing films as shown in FIG. 2 were formed, thermal treatment at 120° C. for 20 min was conducted to completely remove solvent for circumventing possible intermixing between a photoresist system and the catalyst-containing block copolymer thin films. A temperature of 120° C. was chosen because it is well above the boiling temperature of toluene but below the order-to-disorder transition temperatures for both catalyst-containing diblock copolymer systems. OCG825, a highly sensitive broadband photoresist, was then applied on top of these two ordered block copolymer films respectively at a spin speed of 3000 rpm for 30 s to afford a 1.2 m thick film. An ASML 2540 I-line stepper was used to expose the photoresist. A metal ion-free developer, 1:1 water diluted Arch OPD262, removed the exposed resist to generate resist patterns. Further image transfer was accomplished in an Unaxis SLR 770 inductively coupled plasma (ICP) etcher. A mixture of 1 sccm of Cl₂ and 14 sccm of argon was first introduced to remove the self-assembled catalyst-containing polymer films. After the removal of the polymeric layers, 40 sccm of CF₄ was introduced to etch the SiO₂ underlayer. Finally, 40 sccm of O₂ was used to remove any carbon redeposited on the sidewall. After stripping the photoresist, UV-ozonation converted the catalyst-containing polymer film into either cobalt oxide nanoparticles or iron-containing silica nanostructures on top of the posts (posts and plateaus are used interchangeably). The height images of cobalt oxide nanoparticles and iron-containing silica nanostructures after UV-ozonation were viewed by AFM. Following thermolysis at 700° C. in air, carbon nanotube growth was carried out in a CVD system. The substrates were heated to 900° C. under 500 sccm of H₂. Subsequently, a mixture of 800 sccm of CH₄ and 20 sccm of C₂H₄ was added to the gas flow to initiate carbon nanotube growth for the iron-based catalyst system. Only CH₄ was used to initiate CNTs on cobalt nanoparticles. The growth time was 10 min. After the growth, the feed of hydrocarbon gases was switched off and the furnace was cooled to room temperature under protection of H₂.

Raman analysis was used to examine Raman signal intensity of free-standing, or suspended, carbon nanotubes and the CNTs attached onto a surface. The Raman signal intensity of the CNTs that have fallen in the silicon trench region and on the surface of the silicon support structure is lower than the Raman signal intensity of the free-standing CNTs that span the trench, indicating that the greatly enhanced Raman signal is produced from free-standing CNTs (FIG. 4). This suggests that the suspended tubes are free of Van der Waals interaction with the substrate. For CNTs attached on the post, the interaction of CNTs with the underlying surface may impede the radially mechanical oscillation of CNTs, thus yielding weaker Raman signals. FIG. 1 is a side-by-side comparison of Raman spectra of CNTs produced from iron-containing nanostructures. This result further confirms that the absence of substrate interaction results in a greatly enhanced Raman signal. Therefore, they are desirable for accurate determination of tube diameters.

### Example 2

### Generation of a Catalyst-Containing Support Structure for the Synthesis of a 3-Dimensional Network of Suspended CNTs

A Si wafer can be used for the generation of a catalyst-containing support structure for the synthesis of a 3-dimensional network of suspended CNTs. First, pattern the silicon wafer using wet etching methods known in the art (hot KOH, for example). Next, thermally grow a layer of 500 nm thick silicon oxide film on the etched silicon wafer. Remove the thermal oxide growth from the top surface and the bottom of the etched trenches using anisotropic dry etching methods, such as gaseous SF₆, HF or other fluorine or chlorine containing gas. The catalyst deposition can be accomplished using the methods describes in the previous example. Spin-coat cobalt-complexed PS-b-P2VP to form a monolayer of highly ordered cobalt-loaded surface micelles. After coating the PS-b-PFEMS solution, perform a solvent annealing process in toluene to promote the self-assembly process. Once the highly ordered iron-containing films are formed, thermal treatment at 120° C. for 20 min will completely remove the solvent for circumventing possible intermixing between a photoresist system and the catalyst-containing block copolymer thin films. A temperature of 120° C. can be used because it is well above the boiling temperature of toluene but below the order-to-disorder transition temperatures for both catalyst-containing diblock copolymer systems. Carbon nanotube growth can then be accomplishing using the methods described in Example 1.

### Example 3

### Non-Covalent Modification of Suspended CNTs with Biotin

Non-covalent methods of CNT functionalization for the specific binding of proteins, for example, can be accomplished (Chen, et al. (2003) PNAS 100:9 4984-4989). Suspended carbon nanotubes synthesized as described in Example 1 can be modified with a receptor such as Biotin, by the irreversible absorption of modified fatty-acid derivatives to the surface of the suspended CNT. First, activate a suitable fatty-acid derivative, such as polysorbate 20 (Tween 20), for conjugation to receptor by the following method. React Tween 20 with a coupling agent, such as 1,1-carbonyldiimidazole (CU), in dry DMSO at 40° C. for 2 h with stirring. Add diethyl ether to precipitate the activated copolymer. Collect the precipitates, redissolve the solid in DMSO, and reprecipitate using diethyl ether. Dry precipitate in vacuo overnight. To attach Biotin to the activated surface modified CNT, expose the suspended nanotubes to the CDI-activated copolymer in water for 30 min. Rinse with water to remove excess reagent and expose to Biotin-long chain-PEO-amine in a sodium carbonate buffer (pH 9.5) for 24 h at room temperature.

### Example 4

### Non-Covalent Modification of Suspended CNTs with U1A

U1A RNA splicing factor is a prominent autoantigen target in systemic lupus erythematosus (a disorder characterized by skin inflammation) and mixed connective tissue disease, and the detection of autoantibodies directed against this protein forms the basis for a commonly used clinical assay.

Non-covalent methods of CNT functionalization for the specific binding of analytes, such as 10E3, can be accomplished by the use of U1A as a receptor (Chen, et al. (2003) PNAS 100:9 4984-4989). Suspended carbon nanotubes synthesized as described in Example 1 can be modified with a receptor such as U1A by the irreversible absorption of modified fatty-acid derivatives to the surface of the suspended CNT. First, activate a suitable fatty-acid derivative, such as polysorbate 20 (Tween 20), for conjugation to receptor by the following method. React Tween 20 with a coupling agent, such as 1,1-carbonyldiimidazole (CU), in dry DMSO at 40° C. for 2 h with stirring. Add diethyl ether to precipitate the activated copolymer. Collect the precipitates, redissolve the solid in DMSO, and reprecipitate using diethyl ether. Dry precipitate in vacuo overnight. To attach U1A to the activated surface modified CNT, expose the suspended nanotubes to the CDI-activated copolymer in water for 30 min. Rinse with water to remove excess reagent and expose to U1A in a sodium carbonate buffer (pH 9.5) for 24 h at room temperature.

### Example 5

### Non-Covalent Modification of Suspended CNTs with DNA

Non-covalent methods of CNT functionalization for the specific binding of oligonucleotides can be accomplished by the adsorption of single stranded DNA on the surface of the suspended CNTs (Jeng, et al. (2006) Nano Lett. 6:3 371-375). Suspended carbon nanotubes synthesized as described in Example 1 can be modified with single stranded DNA by the following method. First, expose the suspended CNTs to a buffer solution of DNA using a low molecular weight (12-14 KDa) dialysis membrane which allows solvent but not DNA to pass through in order to effectively promote the adsorption of the DNA on the surface of the CNTs. Any free DNA in solution can then be removed using high molecular weight dialysis.

### Example 6

### Modification of Suspended CNTs for the Covalent Attachment of Receptor

Covalent methods of CNT functionalization for the attachment of a desired receptor can be accomplished by appending a reactive functional group, such as a carboxylic acid, to the surface of a suspended nanotube (Wang, et al. (2005) Chem. Phys. Lett. 402: 96-101). First, reflux in 4 M HNO₃ for 24 hours. Remove the HNO₃ and sonicate the CNTs in 1 M HCl for half an hour for the generation of —COOH groups on the surface of the suspended CNTs. Then, wash the carboxylated nanotubes with deionized water and air dry. A desired receptor can then be covalently attached to the functionalized suspended nanotube using standard coupling agents, such as 1,1-carbonyldiimidazole (CDI).

### Example 7

### Modification of Suspended CNTs for the Covalent Attachment of Receptor

In addition to the method described in Example 6, a carboxylic acid can be appended to the surface of a suspended nanotube by the following method (Wang, et al. (2005) Chem. Phys. Lett. 402: 96-101). Sonicate the suspended nanotubes in a 3:1 mixture of concentrated H₂SO₄ and HNO₃ at room temperature for 1-2 hours. Then, sonicate in 1 M HCl for half an hour. Wash the carboxylated nanotubes with deionized water and air dry. A desired receptor can then be covalently attached to the functionalized suspended nanotube using standard coupling agents, such as 1,1-carbonyldiimidazole (CDI).

### Example 8

### Modification of Suspended CNTs for the Covalent Attachment of Receptor

Further to the methods described in Examples 6 and 7, the carboxillic acid functionality can be modified to yield acylchlorides on the surface of the suspended CNTs by the following method (Wang, et al. (2005) Chem. Phys. Lett. 402: 96-101). Stir the carboxylated nanotubes in a 20:1 mixture of thionyl chloride and DMF at 70° C. for 24 hours. After the acylchlorination, wash the nanotubes with anhydrous THF and dry under vacuum at room temperature for about 20 minutes. Receptor attachment can then be carried out in a solvent such as DMF at an elevated temperature for five days. Excess receptor can be washed away using fresh DMF, phosphate buffer and deionized water followed by overnight drying under vacuum.

Although preferred embodiments of the invention are described herein in detail, it will be understood by those skilled in the art that variations may be made thereto without departing from the spirit of the invention or the scope of the appended claims.

What is claimed is:

1. A molecular sensor comprising at least one carbon nanotube as a transducer, based on measuring delta of atomic vibration of Raman sensitive modes upon environmental perturbation, wherein a receptor is first attached to the surface of said carbon nanotube.

2. A molecular sensor comprising a 3-dimensional suspended carbon nanotube network, thereby improving the detection capability of said sensor.

3. The molecular sensor of claim 1 or 2, comprising a plurality of nanotubes.

4. The molecular sensor of claim 1 or 2, further comprising at least one receptor attached to the surface of the nanotube.

5. The molecular sensor of claim 3, further comprising at least one receptor attached to the surface of the nanotube.

6. A solid support having an indentation with walls substantially perpendicular to the surface of the solid support, the substantially perpendicular walls being coated with a solid oxidic layer and over-coated with a layer of a catalyst-particle-containing polymer.

7. The solid support of claim 6, wherein the indentation has a bottom surface substantially parallel to the surface of the solid support, which bottom surface is not coated with the solid oxidic layer.

8. The solid support of claim 6, wherein the indentation has a bottom surface substantially parallel to the surface of the solid support, which bottom surface comprises a growth inhibit layer comprising Si or Si₃N₄.

9. The solid support of claim 6, wherein the catalyst is a particulate carbon-nanotube-growth-promoting catalyst.

10. The solid support of claim 9, wherein the catalyst is an iron- or cobalt-containing catalyst, or a mixture thereof.

11. An oriented carbon-nanotube-containing-solid comprising the solid support system of claim 6 and a plurality of carbon nanotubes grown in situ on the particles of the carbon-nanotube-growth-promoting catalyst.

12. The oriented carbon-nanotube-containing-solid of claim 11, wherein the plurality of carbon nanotubes are grown in situ on the particles of the carbon-nanotube-promoting catalyst present in the layer of catalyst on the substantially perpendicular wall of the indentations.

13. The oriented carbon-nanotube-containing-solid of claim 6 or 11, wherein at least a portion of the carbon nanotubes bridge the substantially perpendicular walls of an indentation.

14. A molecular sensor comprising the oriented carbon nanotube containing solid of claim 6 or 11 and at least one receptor attached to a surface of the carbon nanotubes.

15. A method of detecting analyte in a sample, comprising contacting a sample suspected of containing the analyte with the molecular sensor of claim 4 under suitable conditions that favor binding of analyte to the receptor and detecting analyte bound to the receptor.

15. A method of detecting analyte in a sample, comprising contacting a sample suspected of containing the analyte with the molecular sensor of claim 5 under suitable conditions that favor binding of analyte to the receptor and detecting analyte bound to the receptor.

16. A method of detecting analyte in a sample, comprising contacting a sample suspected of containing the analyte with the molecular sensor of claim 14 under suitable conditions that favor binding of analyte to the receptor and detecting analyte bound to the receptor.

17. A method for preparing the orientated carbon-nanotube-containing solid of claim 11, comprising the steps of: a) depositing a solid oxidic layer onto a solid support having an indentation;
b) depositing a layer of catalyst-particle-containing polymer; and
c) producing a plurality of nanotubes wherein at least a portion of the nanotubes bridge the indentation.

18. The method of claim 17, wherein the solid support comprises a silicon wafer.

19. The method of claim 17, wherein the producing a plurality of nanotubes comprises carbon vapor deposition.

20. A method for preparing the molecular sensor of claim 4, comprising the steps of:
a) depositing a solid oxidic layer onto a solid support having an indentation;
b) depositing a layer of catalyst-particle-containing polymer; and
c) producing a plurality of nanotubes wherein at least one nanotube bridges the indentation; and
d) attaching a receptor to the surface of the at least one nanotube bridging the indentation.
